(19) United States
(12) Patent Application Publication
(10) Pub. No.: US 2008/0275401 A1
(43) Pub. Date: Nov. 6, 2008

(54) Catheter anchor and system/method regarding same

(76) Inventors: Shahn S. Sage, Andover, MN (US); Michael Kern, Minneapolis, MN (US)

Correspondence Address: MUETING, RAASCH & GEBHARDT, P.A., P.O. BOX 581336, MINNEAPOLIS, MN, 55458-1336, US

(21) Appl. No.: 11/799,166

(22) Filed: May 1, 2007

(51) Int. Cl. A61M 25/02

(52) U.S. Cl. 604/175

(57) ABSTRACT

An anchor for use in an anchor system and method for securing a catheter to tissue includes a flexible body portion that defines one or more openings through which a catheter is positioned when secured to the tissue. For example, the one or more openings may be a channel through the anchor having a diameter that is less than the outer diameter of the catheter.

### TECHNICAL FIELD

The present invention relates generally to anchors for securing catheters (e.g., catheters positioned for delivering drug or medication to a treatment site in a patient's body).

### BACKGROUND

Medication delivery to the brain, spinal cord, cerebrospinal fluid, or other tissue throughout the body is useful in treating many diseases and illnesses. For example, through use of an implantable pump and catheter, precise drug doses can be directly delivered to an area of interest to treat the disease or illness, or its symptoms.

Use of a catheter to deliver a therapeutic agent to a target site generally involves the insertion of the catheter into the body and dispensing the agent at the desired location or target site. During a typical procedure, an incision may be made, a needle may be inserted (e.g., to access the target such as cerebrospinal fluid), a catheter may be inserted through the needle to the target site, and the catheter then anchored to reduce the chance of the catheter moving.

As one can appreciate, precisely locating an inserted device such as a catheter is important to ensure that the therapeutic substance is provided to the desired target site. Depending on the particular treatment administered, a tip of the inserted device may need to be located within a relatively narrow tolerance range of the target site. Even minimal variation of the tip from the target site may result in unsatisfactory therapeutic results. Accordingly, reliable methods and apparatus for locating the device and maintaining the device at the location are needed.

In other words, in view of the desirability of providing a drug or medicament at a specific target site, movement of the catheter from its desired position is problematic. This problem is sometimes referred to as dislodgement. The term dislodgement may be used to describe a situation where the distal tip of the catheter is moved enough from its desired position for the therapy to be affected. For some therapies (e.g., intrathecal administration of drugs to the spinal cord) just a small movement (e.g., less than one or two centimeters) may cause a noticeable decrease in the effectiveness of the therapy. For other types of therapies, larger movements are needed to decrease the effectiveness of therapy, but location is still important. Still further, applying a drug or a medicament to a site different than the target site may result in undesirable effects. Therefore, dislodgement is a condition to be avoided.

There are various ways that have been described to attempt to reduce the problem of dislodgement (e.g., movement of the catheter tip). For example, one technique is to suture the catheter to tissue to anchor the catheter to the tissue. However, this approach may result in the suture cutting through or occluding the catheter, thereby disabling the catheter (e.g., preventing drug from passing to the distal end of the catheter and/or allowing for leakage out of the cut area to a site that is not to be treated).

Yet further, for example, another technique of reducing the chance of dislodgement is to associate an anchor with the catheter and then secure (e.g., suture) the anchor structure to tissue. In such a manner, the anchor reduces the chance of the catheter moving as the likelihood of the anchor itself moving is reduced.

Examples of one or more anchors for use in securing a catheter are described in U.S. Pat. No. 6,554,802 issued 29 Apr. 2003 to Pearson et al., and entitled “Medical Catheter Anchor.” For example, one anchor described therein includes a generally tubular body including a lumen for receiving the catheter and a pair of wings to assist in securing the device. The tubular body has a slot that extends through the body between the wings. The wings and the slot cooperate so that the opposed edges of the slot come together as the wings are brought into contact with each other. The wings interact with the slot to radially compress the catheter within the lumen of the tubular body to hold the catheter in a fixed relation thereto. The wings when brought into contact with each other provide an eyelet for use in suturing the anchor, for example, to tissue.

Although previously described anchors provide for the reduction in the likelihood of dislodgement, movement of the catheter may still occur even though such anchors attempt to secure and fix the anchors relative to tissue. For example, there may still be a chance of slippage through the anchor.

Further, for example, even though it is less likely for the anchor to substantially move relative to the tissue, even small amounts of movement by the anchor (e.g., rocking of the anchor described in U.S. Pat. No. 6,554,802 about the single suture point thereof) may cause movement of the catheter over time. For example, over time, even if the catheter does not move relative to the anchor, any movement of the anchor (e.g., rocking thereof) may cause the distal end of the catheter to leave the target site and, in one or more cases, bunch up and/or coil at a location between the anchor and the target site.

### SUMMARY

The present invention may overcome these and other issues with currently described anchors by providing a reduction in the likelihood of the catheter from moving after being positioned using one or more embodiments of an anchor, an anchor system, or an anchor method such as that described herein.

An anchor system according to one embodiment of the present invention for use in securing a catheter to tissue includes a catheter anchor placement tool and an anchor. The catheter anchor placement tool (e.g., a needle device or a tool having a cylindrical portion) includes an elongate body portion, wherein the elongate body portion includes a non-flexible outer cylindrical surface located between a first end and a second end of the elongate body portion. Further, the elongate body portion defines a lumen through the elongate body portion from the first end to the second end having a diameter that is greater than an outer diameter of the catheter to allow the catheter to pass through the catheter anchor placement tool. The anchor includes a body member formed of a flexible material defining at least one opening through which the catheter is positioned when secured using the anchor. The outer cylindrical surface of the catheter anchor placement tool is configured so as to hold the anchor thereon such that the inner diameter of the at least one opening defined through the anchor is greater than the outer diameter of the catheter when the anchor is positioned on the outer cylindrical surface.

In one embodiment of the system, the body member of the anchor includes an elongate body member that defines a channel extending through the elongate body member from a first end to a second end thereof, wherein the cross-sectional inner diameter along at least a portion of the length of the channel is less than the outer diameter of the catheter when in a normal state. The outer cylindrical surface of the catheter anchor placement tool has an outer diameter sized to hold the anchor thereon in a flexed state such that the cross-sectional inner diameter of at least a portion of the channel is greater than the outer diameter of the catheter when the anchor is positioned on the outer cylindrical surface.

In another embodiment of the system, the anchor further includes at least one suture eyelet containing element extending from the body member. The at least one suture eyelet containing element defines at least two suture eyelets. Each of the at least two suture eyelets is offset from a centerline extending through the anchor to provide at least two separate suture points offset from the centerline for suturing the anchor to tissue.

Another anchor according to one embodiment of the present invention for use in securing a catheter to tissue includes an elongate body member. A channel is defined through the elongate body member from a first end to a second end thereof. The cross-sectional inner diameter along at least a portion of the length of the channel is less than the outer diameter of the catheter when the anchor is in a normal state preventing the insertion of the catheter through the channel. Further, at least a portion of the anchor is formed of a flexible material that allows the cross-sectional inner diameter along the portion of the length of the channel (e.g., along a part or the entire length of the channel) to be expanded to a cross-sectional inner diameter greater than the outer diameter of the catheter when the anchor is in a flexed state.

Another anchor according to one embodiment of the present invention for use in securing a catheter to tissue includes an elongate body member, wherein a channel is defined through the elongate body member from a first end to a second end thereof. The elongate body member includes a slot that extends from an outer surface of the elongate body member to the channel defined thereby (e.g., the slot having side edges opposed to each other). The anchor further includes a pair of wings extending from the elongate body member on opposite sides of the slot. The pair of wings are separated from each other by an angle. The wings and the slot cooperate so that when the wings are brought into contact with each other the anchor secures the catheter in the channel and the pair of wings defines a first suture eyelet offset from a centerline of the channel when the pair of wings are brought into contact with each other. Yet further, the anchor includes an additional wing extending from the elongate body member defining an additional suture eyelet offset from the centerline of the channel.

A method of securing a catheter to tissue according to one embodiment of the present invention includes providing an anchor comprising a body member, wherein the body member comprises a flexible material defining at least one opening through the body member. A non-flexible portion of a catheter anchor placement tool is inserted into the at least one opening of the anchor, wherein the anchor is held thereon. The method further includes inserting the catheter through a lumen in the catheter placement tool. The anchor is held and the catheter anchor placement tool is removed so as to position the anchor on the catheter (e.g., so as to provide a compression fit of the anchor on the catheter).

In one embodiment of the method, the method further includes making an incision in a tissue region and inserting a portion of the anchor at least 1 cm into the tissue. Further, the portion of the anchor may be inserted at least 2 cm into the tissue.

In another embodiment of the method, inserting a catheter anchor placement tool into the at least one opening includes loading the anchor onto a needle device, wherein the needle device includes a needle shaft having an outer cylindrical surface and defining at least a portion of a lumen thereof. The needle shaft terminates in a needle tip for insertion into the body of a patient. Further, prior to inserting the catheter through the lumen, the method further includes making an incision in a region of the patient and inserting the needle device with the anchor loaded thereon through the incision.

In yet another embodiment of the method, inserting a catheter anchor placement tool into the channel of the anchor includes loading the anchor onto a cylindrical portion of the catheter anchor placement tool defining at least a portion of a lumen. The method further includes providing a needle device comprising a needle shaft defining a lumen through the needle and terminating at a needle tip for insertion into the body of a patient and inserting the needle device into the patient, wherein the catheter is inserted through the lumen in the needle device and positioned in the patient. The needle device is then removed from the patient. Thereafter, an incision in the patient is made proximate the positioned catheter such that the catheter can be inserted into the lumen of the catheter placement tool and the anchor positioned on the catheter at a desired location.

The above summary is not intended to describe each embodiment or every implementation of the present invention. Rather, a more complete understanding of the invention will become apparent and appreciated by reference to the following Detailed Description of Exemplary Embodiments and claims in view of the accompanying figures of the drawing.

### BRIEF DESCRIPTION OF THE VIEWS OF THE DRAWING

The present invention will be further described with reference to the figures of the drawing, wherein:

FIGS. 1A-1E illustrate one embodiment of a method and/or anchor system used to position an anchor for securing a catheter according to the present the invention;

FIGS. 2A-2C show a first front perspective view, a second rear perspective view, and a perspective cross-section view taken along line C-C of FIG. 2A, respectively, of one embodiment of the anchor used for securing a catheter as shown in FIGS. 1A-1E according to the present invention;

FIGS. 3A-3D illustrate one embodiment of another method and/or anchor system used to position an anchor for securing a catheter according to the present the invention;

FIG. 4 is a perspective cross-section view taken along line 4-4 of FIG. 3D of one embodiment of a catheter anchor placement tool used in the process of securing a catheter as shown in FIGS. 3A-3D according to the present invention;

FIGS. 5A-5C show a perspective view, an end view, and a perspective cross-section view taken along line C-C of FIG. 5A, respectively, of one embodiment of the anchor used for securing a catheter as shown in FIGS. 3A-3D according to the present invention;

FIGS. 6A-6F show a perspective view of another embodiment of an anchor according to the present invention, a side view of the anchor in a normal state, an end view of the anchor in a normal state, a cross-section view taken along line D-D of FIG. 6C, a side view of the anchor in a compression state, and a perspective view of the anchor in a compression state sutured to tissue, respectively.

FIGS. 7A-7C illustrate one embodiment of another method and/or anchor system used to position the anchor illustrated in FIGS. 6A-6F for securing a catheter according to the present the invention;

FIGS. 8A-8D show a plan view of the anchor in a normal state, an end view of the anchor in a normal state, a cross-section view taken along line C-C of FIG. 8A, and a more detailed view of section D of FIG. 8C, respectively.

FIGS. 9A-9C illustrate one embodiment of yet another anchor, method, and anchor system used to position the anchor, wherein FIGS. 9A and 9B illustrate use of a tool to position the anchor and having the anchor loaded thereon in a flexed state, and FIG. 9C shows the anchor in a normal state securing the catheter therein.

The figures are rendered primarily for clarity and, as a result, are not necessarily drawn to scale.

### DETAILED DESCRIPTION OF EXEMPLARY EMBODIMENTS

In the following detailed description of illustrative embodiments of the invention, reference is made to the accompanying figures of the drawing which form a part hereof, and in which are shown, by way of illustration, specific embodiments in which the invention may be practiced. It is to be understood that other embodiments may be utilized and structural changes may be made without departing from the scope of the present invention.

The instant invention is directed to medical systems and devices, as well as to procedures for using the same. For instance, one embodiment of the invention may be directed to an anchor system or kit for precisely positioning and securing a tip of a medical device such as a catheter using an anchor. In the illustrated embodiment, exemplary systems and methods are described and illustrated in the context of securing a catheter having a distal tip at a target site (e.g., such as securing an anchor for the catheter at a desired position in tissue wherever found in the body, such as in epidural or intrathecal space of a spinal cord or brain). However, such anchors may be used to secure other devices without departing from the scope of the invention, and catheter as used herein shall refer to any device (e.g., such as spinal cord stimulation leads, electrodes including an electrode tip to be positioned at a treatment site, or any other stimulation leads) that could be suitably positioned and secured using an anchor as described in one or more embodiments herein.

The present invention employs one or more techniques as shall be described in detail herein to secure a catheter using an anchor. For example, in one or more embodiments such techniques may include the use of a self gripping function for securing the catheter in a channel or one or more openings of an anchor, may include an anchor of which a portion thereof may be inserted into tissue of the body more than one centimeter (and even more than two centimeters) to provide a more secure catheter, may include an anchor that has two or more suture locations (e.g., that are offset from a centerline of a channel defined by the anchor) to prevent rocking of the anchor which may occur when only a single suture point is employed, and may include a catheter anchor placement tool formed of a non-flexible material upon which an anchor may be loaded and through which a catheter may be inserted for positioning the anchor on the catheter in the patient (e.g., easing the placement of a flexible anchor on the catheter).

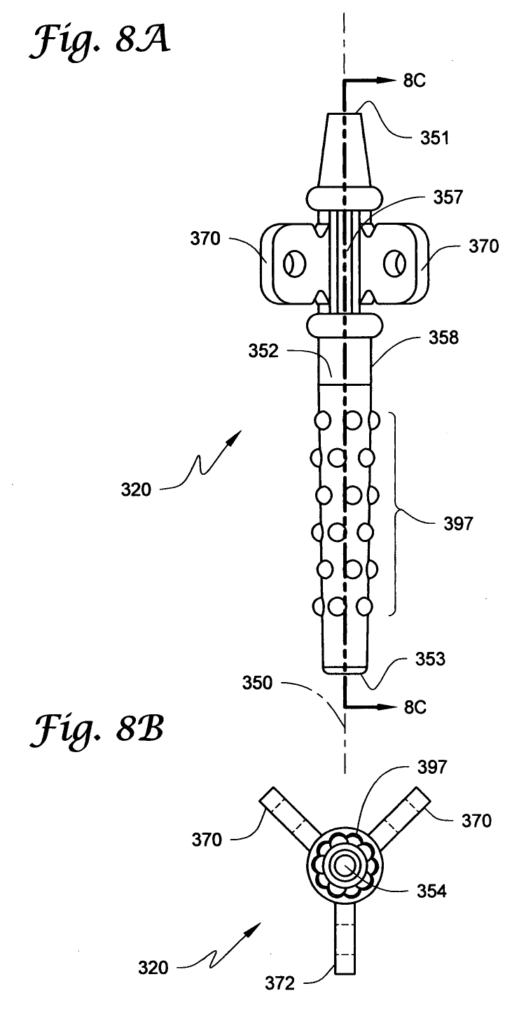

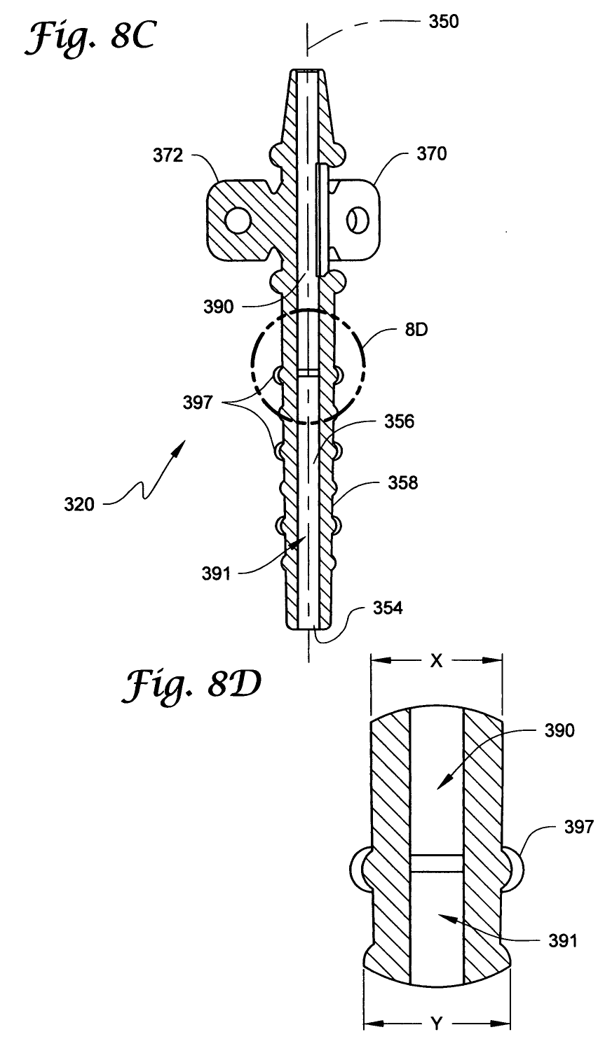

It will be recognized from the description provided herein that one or more of the features described herein with reference to any particular embodiment may be used in combination with one or more features of any other embodiment, even though such combinations are not explicitly provided herein. For example, the stability structure described with reference to FIG. 8 can be used with any other anchors described herein. Further, for example, position of suture eyelets of one embodiment may be used in the anchor of any other embodiment. Any catheter configuration may be suitably anchored using one or more of the anchors described herein, including those used for electrical stimulation therapy, as well as delivering drugs or medication. As such, the present invention is not limited to only the combinations specifically described.

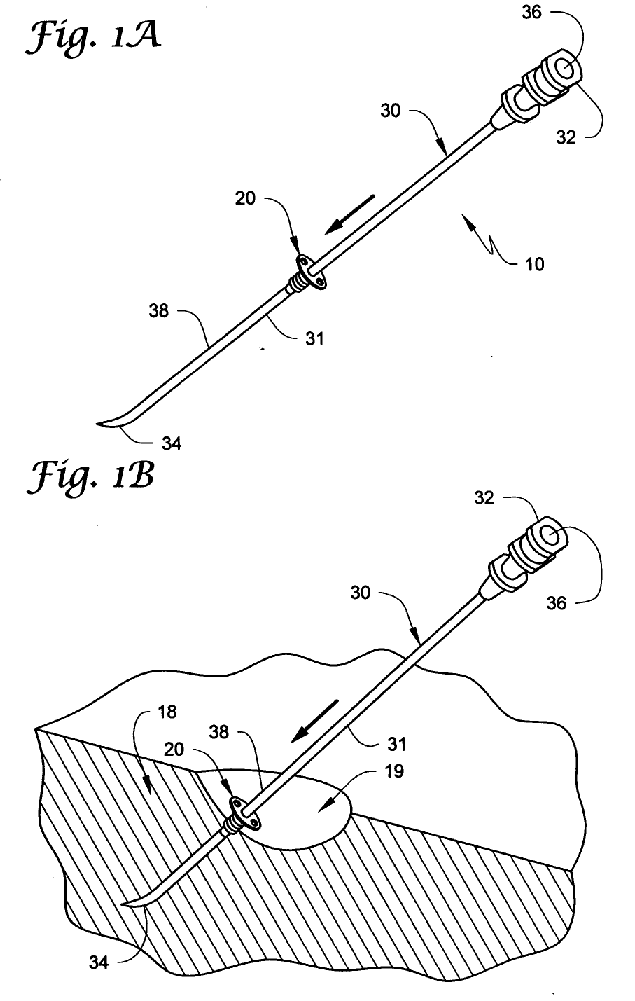

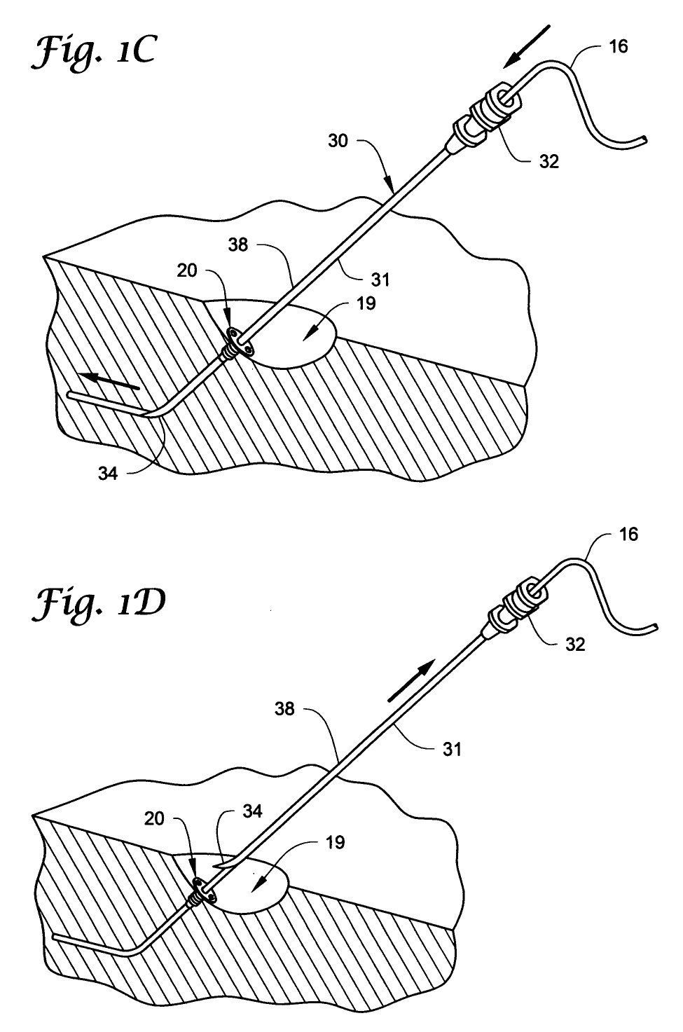

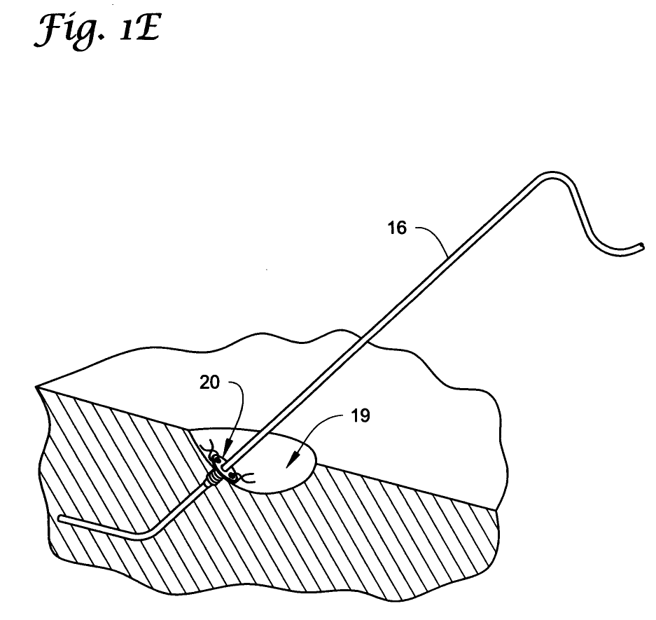

FIGS. 1A-1E show one illustrative embodiment of a method that uses an anchor system 10 for securing a catheter 16 relative to tissue of a patient. The anchor system 10 includes an anchor 20 that may be used to secure the catheter 16 to tissue. One will recognize that any number of different anchors may be used to secure the anchor according to the method illustrated in FIGS. 1A-1E and the present invention is not limited to the use of any particular anchor described herein with any particular method of using such an anchor.

The catheter 16, at least in one embodiment as shown in FIG. 1C, is generally a flexible tube having a generally consistent outer diameter along the length of the catheter 16. For example, the catheter may be a thin flexible tube for insertion into a part of the body to inject or drain away fluid, or to keep a passage open. The catheter 16 includes a lumen extending along the length thereof.

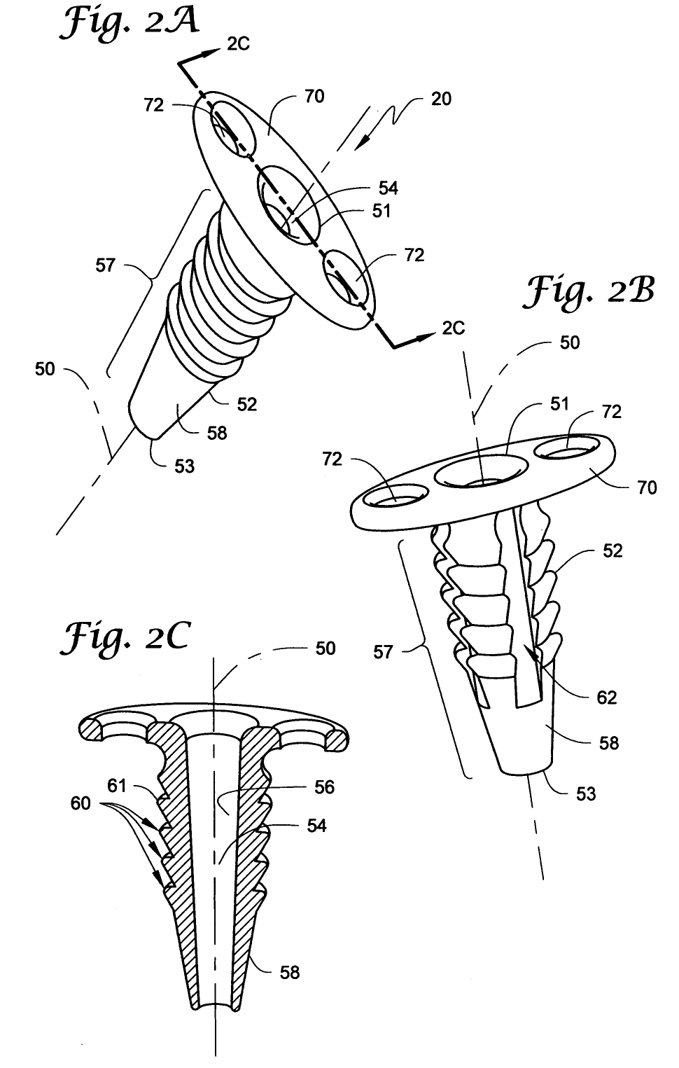

The anchor 20 is shown in further detail in FIGS. 2A-2C. FIG. 2A shows a front perspective view of the anchor 20. FIG. 2B shows a rear perspective view of the anchor 20 and FIG. 2C shows a perspective cross-section view taken along line C-C of FIG. 2A. The anchor 20 includes an elongated body member 52 that extends between a first end 51 and a second end 53 along a centerline 50 of the anchor 20. At least in one embodiment, when the anchor 20 is in a normal state (i.e., not flexed or expanded in any particular orientation or direction), the centerline 50 corresponds to a straight axis along which the elongated body member 52 extends.

The elongated body member 52 includes an inner surface 56 at a radial distance from the centerline 50 that defines a channel 54 from the first end 51 to the second end 53 along the centerline 50. The catheter 16 is positioned and secured to the anchor 20 within the channel 54 as will be further described herein. At least in one embodiment, when the anchor 20 is in a normal state (i.e., not flexed or expanded in any particular direction or orientation), the channel 54 takes the form of a cylindrical channel. In at least one embodiment, the channel 54 has a cross-sectional inner diameter along at least a portion of the centerline 50 that is less than the outer diameter of the catheter 16. In other words, at least in one embodiment, the catheter 16 cannot be inserted through the channel 54 defined by the anchor 20 when the anchor 20 is in a normal state. For example, the inner diameter of the channel 54 may be 20 percent to 50 percent smaller than the outer diameter of the catheter 16.

The anchor 20 and the other anchors described herein are formed of a flexible material. For example, the anchors may be molded of silicone or any other flexible polymeric material such as urethane. Further, for example, any other flexible biocompatible material may be used to form the anchors, such as a memory material (e.g., Nitinol wire or band), or any other material that is able to change its original shape (e.g., to a flexed state) and return to the original shape (e.g., to a normal state). One will recognize from the description herein that, at least in the embodiment described with reference to FIG. 2, any flexible material suitable to allow expansion of the diameter of channel 54 in the manner described herein (e.g., when loaded on an anchor placement tool in a flexed state), and which allows subsequent return to its normal state, may be used according to the present invention.

Further, the anchor 20 includes a suture eyelet containing element 70 radially extending from the elongate body member 52 at the first end 51 of the elongate body member 52. In other words, the suture eyelet containing element 70 is located in a plane orthogonal to the centerline 50 of the anchor 20. The suture eyelet containing element 70 defines two suture eyelets 72 (e.g., suture rings or openings) that are each offset from the centerline 50 of the anchor 20. As shown in FIG. 2, each of the suture eyelets 72 lie in a plane that is orthogonal to the centerline extending through the anchor 20. In such an orientation, suturing of the anchor 20 to tissue is performed in a generally parallel manner relative to the centerline 50 of the channel 54.

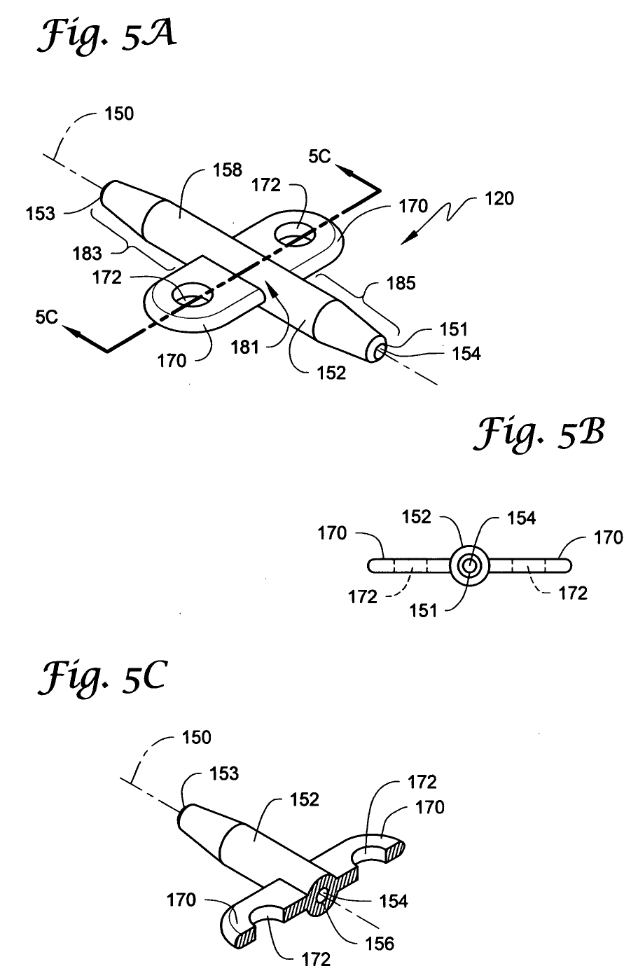

Although the anchor 20 of FIG. 2 shows the suture eyelets 72 oriented such that they lie in a plane generally orthogonal to the centerline 50, one skilled in the art from the description herein will recognize that the placement of the suture eyelets (e.g., location points for suturing) may be provided at any location as long as such locations are offset from the centerline of the anchor. In such a manner, the anchor when sutured to the tissue is prevented from rocking and maintains the catheter in a stable position. For example, as shown in FIG. 5, the suture eyelets lie in a plane that is generally parallel to the centerline and in a center region of the anchor. Further, such suture eyelets do not need to be symmetrically arranged with respect to the body member of the anchor, or located opposite one another relative to the body member. One will recognize that the further apart the suture points, the more stable the anchor will be relative to the tissue. However, this is balanced by the need to keep the anchor small. Further, any number of suture eyelets may be provided and the present invention is not limited to just two suture eyelets (or suture points).

The elongated body member 52 includes at least a portion 57 thereof along the length of the anchor 20 that is insertable into the tissue of the patient. The length (i.e., measured along the centerline 50) of the portion 57 insertable into the tissue of the patient extending from the suture eyelet containing element 70 to the second end 53 of the anchor 20 is, at least in one embodiment, greater than one (1) centimeter. In other words, from the point where the suture eyelet containing element 70 prevents insertion into the tissue to the second end 53 is greater than one centimeter. By providing such a length, stabilization of the anchor 20 within the tissue is further enhanced. Still further, such a length that may be inserted may even be greater than two (2) centimeters.

Still further, the elongated body member 52 of the anchor 20 includes an outer surface 58 that may include any number of different stability structures suitable for increasing the stability of anchor 20 when affixed to tissue. For example, such stability structures may include tines 62 extending along the length of the outer surface 58 of the elongated body member 52. Such tines may take any number of different shapes and number, and the present invention is not limited to only the illustrated views provided herein. Further, for example, such stability structures may include barbs 60 (e.g., elements with backward facing points 61) to reduce the likelihood of the anchor 20 withdrawing from the tissue once inserted. Such barbs may take the form of any number of different shapes and number, and the present invention is not limited to only the illustrated views provided herein. Further, any number of other types of stability structures may be employed including, for example, bumps, texture, or any other friction based shapes that would provide for stability of the anchor in the tissue.

With further reference to the anchor 20 shown in FIG. 2, and FIGS. 1A-1E which show one illustrative embodiment of using this anchor 20 to secure catheter 16 relative to tissue 18 of a patient, the anchor system 10 shall be further described. In addition to the anchor 20, the anchor system 10 further includes a catheter anchor placement tool, or in this particular embodiment, namely a needle device 30. For example, any needle device suitable for placement of a catheter may be used. Such needle devices are available, for example, from EpiMed International (Johnstown, N.Y.). For example, such needle devices are available in various sizes (e.g., 3½ inch, 4 inch, and 6 inch needles).

Generally, the needle device 30 includes an elongate body member in the form of a non-flexible needle shaft 31 extending from an attachment end structure 32 and which terminates in a needle tip 34. The attachment end structure 32 will vary depending on the application for which the needle device 30 is employed and the needle tip 34 is configured to be inserted into the body of a patient (e.g., epidural needles, spinal needles, etc.)

The non-flexible needle shaft 31, for example, may be formed of stainless steel. However, any material providing for a non-flexible shaft upon which the anchor 20 can be loaded may be used. As the anchor 20 is formed of a flexible material, by providing the anchor 20 on a non-flexible component (e.g., a component having less flexibility than the anchor), securing the anchor 20 to the catheter 16 is made easier as the needle shaft (e.g., the catheter anchor placement tool) provides some support structure for the anchor when it is loaded thereon.

The needle shaft 31 defines a lumen 36 between the attachment end 32 and the needle tip 34. The lumen has a diameter that is greater than an outer diameter of the catheter 16 such that the catheter is allowed to pass through the needle device 30 (e.g., such that the tip of the catheter 16 can be place at a target site, such as that corresponding to the location of the tip 34 of the needle when inserted into a patient).

The needle shaft 31 further includes an outer cylindrical surface 38 located between the attachment end 32 and the needle tip 34. The outer cylindrical surface 38 may be along substantially the entire length of the needle device or a certain defined length thereof. At least in one embodiment, the outer cylindrical surface 38 has an outer diameter that is greater than the cross-sectional inner diameter of the channel 54 defined by the elongated body member 52 of the anchor 20.

As shown in FIG. 1A, the needle device 30 is inserted in the channel 54 defined in the anchor 20. With the outer diameter of the outer cylindrical surface 38 being greater than the cross-sectional inner diameter of the channel 54, the flexible anchor is expanded into a flexed state (e.g., an expanded diameter state, different than the normal diameter state of the anchor). In other words, the cross-sectional inner diameter of at least a portion of the channel 54 is increased such that it is greater than the outer diameter of the catheter 16 when the anchor 20 is positioned on the outer cylindrical surface 38 of the needle device 30. When reference is made to a flexed state, the component in the flexed state is bent, expanded, or otherwise deformed from a normal state.

With the anchor 20 loaded onto the needle device 30 such that it is in a flexed state, an incision is made to access an incision region 19 (e.g., a tissue region, such as fascia or other connective tissue) of the body in a location where the needle device 30 is to be inserted as shown in FIG. 1B. The needle device 30 with the anchor 20 loaded thereon is then inserted into the body at the incision region 19, as shown in FIG. 1B, with the needle tip 34 being moved or extended to the desired site (e.g., spinal area) which may correspond to where the tip of the catheter 16 is to be positioned.

The catheter 16 may then be inserted into the lumen of the needle device 30 as shown in FIG. 1C and the tip of the catheter 16 positioned as desired at the target site. The anchor 20 is positioned, at least in one embodiment, with at least one centimeter of portion 57 of the anchor 20 extending into the tissue (e.g., not fat cells), such as connective tissue (e.g., fascia). In other embodiments, two or more centimeters of the portion 57 of the anchor 20 extends into the tissue.

The anchor 20 may then be held in the desired place and the needle device 30 withdrawn and removed from the body of the patient as shown in FIG. 1D. As the anchor 20 is removed from the needle device 30, the anchor 20 will tend back to its normal state such that the inner diameter of the channel 54 will decrease, and since the inner diameter of the channel 54 in its normal state is less than the outer diameter of the catheter 16, a compression fit between the anchor 20 and the catheter 16 in the channel 54 is provided. In other words, the anchor 20 is essentially self gripping on the catheter 16 as the anchor returns to the anchor's normal state.

The anchor 20 may then be sutured to connective tissue as shown in FIG. 1E. For example, each of the suture eyelets 70 of the anchor 20 offset from the centerline 50 thereof may be sutured to the connective tissue to secure the anchor 20 to the tissue 18.

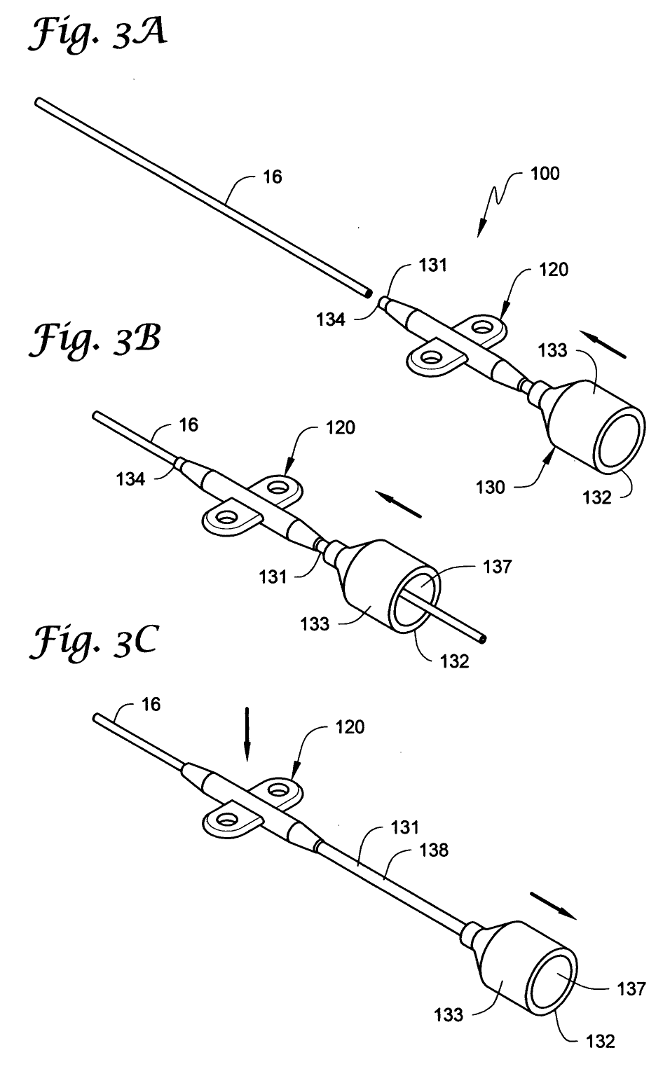

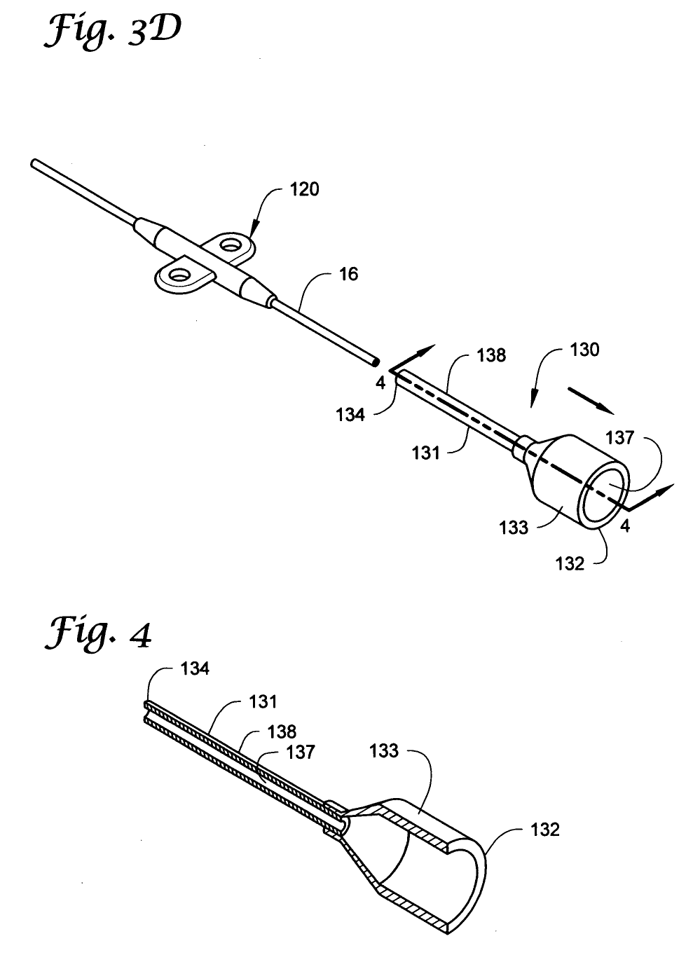

One skilled in the art will recognize from the description herein that various methods of securing the catheter 16 using one or more various anchor configurations according to the present invention, in addition to that described with reference to FIGS. 1 and 2, are possible. For example, FIGS. 3A-3D illustrate another embodiment of a method using an anchor system 100 to secure the catheter 16 relative to tissue of a patient. The anchor system 100 includes another embodiment of an anchor 120 that may be used to secure the catheter 16 to tissue. One will recognize that any number of different anchors may be used to secure the catheter 16 according to the method illustrated in FIGS. 3A-3D.

The anchor 120 is shown in further detail in FIGS. 5A-5C. FIG. 5A shows a perspective view of the anchor 120. FIG. 5B shows an end view of the anchor 20 and FIG. 5C shows a perspective cross-section view taken along line C-C of FIG. 5A. The anchor 120 includes an elongated body member 152 that extends between a first end 151 and a second end 153 along a centerline 150 of the anchor 120. At least in one embodiment, when the anchor 120 is in a normal state (i.e., not flexed or expanded in any particular orientation or direction), the centerline 150 corresponds to a straight axis along which the elongated body member 152 extends.

The elongated body member 152 includes an inner surface 156 at a radial distance from the centerline 150 that defines a channel 154 from the first end 151 to the second end 153 along the centerline 150. The catheter 16 is positioned and secured to the anchor 120 within the channel 154 as will be further described herein. When the anchor 120 is in a normal state (i.e., not flexed or expanded in any particular direction or orientation), the channel 154 takes the form of a cylindrical channel. In at least one embodiment, the channel 154 has a cross-sectional inner diameter along the centerline 1 50 that is less than the outer diameter of the catheter 16. In other words, at least in one embodiment, the catheter 16 cannot be inserted through the channel 154 defined by the anchor 120 when the anchor 120 is in a normal state. For example, the inner diameter of the channel 154 may be 20 percent to 50 percent smaller than the outer diameter of the catheter 16. The anchor 120 is formed of a flexible material such that, at least in the embodiment described with reference to FIG. 5, the flexible material is suitable to allow expansion of the diameter of channel 154 in the manner described herein (e.g., when loaded on an anchor placement tool in a flexed state), and to allow subsequent return to its normal state.

Further, the anchor 120 includes one or more suture eyelet containing elements 170 extending from the elongate body member 152 at a center region 181 of the elongate body member 152. The suture eyelet containing elements 170 are located in a plane parallel to the centerline 150 of the anchor 120 and on opposite sides thereof. The suture eyelet containing elements 170 define two suture eyelets 172 (e.g., suture rings or openings) that are each offset from the centerline 150 of the anchor 120. As shown in FIG. 5, each of the suture eyelets 172 lie in a plane that is parallel to the centerline 150 extending through the anchor 120. In such an orientation, suturing of the anchor 120 to tissue is performed in generally orthogonal manner relative to the centerline 150 of the channel 154. As described with reference to the anchor 20 of FIG. 2, the suture eyelets 170 (e.g., location points for suturing) may be provided at any location as long as such locations are offset from the centerline of the anchor.

The elongated body member 152 includes at least a portion 183 and/or 185 thereof along the length of the anchor 120 that is insertable into the tissue of the patient. The length of the portion 183 and/or 185 insertable into the tissue of the patient (i.e., extending from the suture eyelet containing elements 170 to the first end 151 or second end 53 of the anchor 120) is, at least in one embodiment, greater than one (1) centimeter. Still further, such a length of the anchor 120 that may be inserted into the tissue may be even greater than two (2) centimeters.

Still further, the elongated body member 152 of the anchor 120 includes an outer surface 158 that is tapered to provide body regions that are narrower at the first end 151 and second end 153 relative to the rest of the elongated body member 152. Such tapering eases the insertion force necessary to insert the anchor into the tissue and also relieves strain on the catheter 16 when secured in the anchor 120.

With the suture eyelet containing element 170 extending from the body member 152 at the center region 181 thereof, the two end portions 183, 185 of the elongated body member 152 can be configured in substantially the same manner. For example, as shown in FIG. 5, first end portion 185 between the suture eyelet containing elements 170 and the first end is configured the same as the second end portion 183 of the body member 152 between the suture eyelet containing elements 170 and the second end 153. As the end portion 183 and end portion 185 are identical, a user cannot mistakenly load the anchor 120 on a catheter anchor placement tool incorrectly.

With further reference to the anchor 120 shown in FIG. 5, and FIGS. 3A-3D which show one illustrative embodiment of using this anchor 120 to secure catheter 16 relative to tissue 18 of a patient, the anchor system 10 shall be further described. In addition to the anchor 120, the anchor system 100 further includes a catheter anchor placement tool 130.

Generally, the catheter anchor placement tool 130 includes a cylindrical body portion 131 coupled to a holding portion 133 and defining a lumen 137 therethrough. The non-flexible cylindrical body portion 131 and the holding portion 133 extend between a first end 132 and a second end 134. The holding portion 132 may be of any suitable configuration defining at least a portion of a lumen 137 continuous with the portion of the lumen 137 defined through the cylindrical body portion 131. For example, the holding portion may be a knob having an opening formed therein or any other structure that may be held or grasped by a user.

The cylindrical body portion 131, for example, may be formed of biocompatible materials, e.g., stainless steel, a hard polymer, ceramics, polyetheretherketone (PEEK), etc. However, any material providing for a non-flexible element (e.g., less flexible than the anchor material) upon which the anchor 120 can be loaded may be used. As the anchor 120 is formed of a flexible material, by providing the anchor 120 on a non-flexible component, securing the anchor 120 to the catheter 16 is made easier as the cylindrical body portion 131 of the catheter anchor placement tool 130 provides a support structure to the anchor 120 when it is loaded thereon.

The lumen 137 defined through the catheter placement tool 130 has a diameter that is greater than an outer diameter of the catheter 16 such that the catheter is allowed to pass through the tool 130. The cylindrical body portion 131 further includes an outer cylindrical surface 138 located between the first end 132 and the second end 134. The outer cylindrical surface 138 may be along substantially the entire length of the cylindrical body portion 131 or a certain defined length thereof. At least in one embodiment, the outer cylindrical surface 138 has an outer diameter that is greater than the cross-sectional inner diameter of the channel 154 defined by the elongated body member 152 of the anchor 120.

In this illustrative embodiment of securing the catheter 16, the catheter 16 is positioned in the body as desired prior to positioning of the anchor 120 on the catheter 16, as represented generally by FIG. 3A. For example, at least in one embodiment, a needle device such as that described with reference to FIG. 1, may be inserted into the body at a desired location to position the catheter 16 or the tip thereof at the target site (e.g., spinal area). The catheter 16 may then be inserted into the lumen of the needle device and the tip of the catheter 16 positioned as desired at the target site. The needle device may then be withdrawn and removed from the body of the patient resulting in the catheter 16 being positioned, but unsecured.

Further, as shown in FIG. 1A, the cylindrical body portion 131 that includes the outer cylindrical surface 138 is inserted in the channel 154 defined in the anchor 120. With the outer diameter of the outer cylindrical surface 138 being greater than the cross-sectional inner diameter of the channel 154, the flexible anchor is expanded into a flexed state (e.g., an expanded diameter state, different than the normal diameter state of the anchor). In other words, the cross-sectional inner diameter of at least a portion of the channel 154 is increased such that it is greater than the outer diameter of the catheter 16 when the anchor 120 is positioned on the outer cylindrical surface 138 of the cylindrical body portion 131.

With the anchor 120 loaded onto the cylindrical body portion 131 such that it is in a flexed state, an incision is made proximate the positioned catheter 16 to access an incision region (e.g., a tissue region, such as fascia or other connective tissue) of the body in a location where the anchor 120 is to be secured. The positioned catheter 16 is then inserted into the cylindrical body portion 131 having the anchor 120 loaded thereon as shown in FIG. 3B. The anchor 120 may then be advanced along the length of the catheter 16 by the tool 130 until the desired position for the anchor 120 is reached (e.g., with at least one centimeter of portion 183 or 185 of the anchor 120 extending into the tissue, such as connective tissue (e.g., fascia)).

The anchor 120 may then be held in the desired place and the cylindrical body portion 131 withdrawn and removed from the body of the patient. As the anchor 120 is removed from the cylindrical body portion 131, the anchor 120 will tend back to its normal state such that the inner diameter of the channel 154 will decrease, and since the inner diameter of the channel 154 in its normal state is less than the outer diameter of the catheter 16, a compression fit between the anchor 120 and the catheter 16 in the channel 154 is provided. In other words, the anchor 120 is essentially self gripping on the catheter 16 as the anchor 120 returns to the anchor's normal state.

The anchor 120 may then be sutured to connective tissue at the desired site. For example, each of the suture eyelets 172 of the anchor 120 offset from the centerline 150 thereof may sutured to the connective tissue to secure the anchor 120 to the tissue.

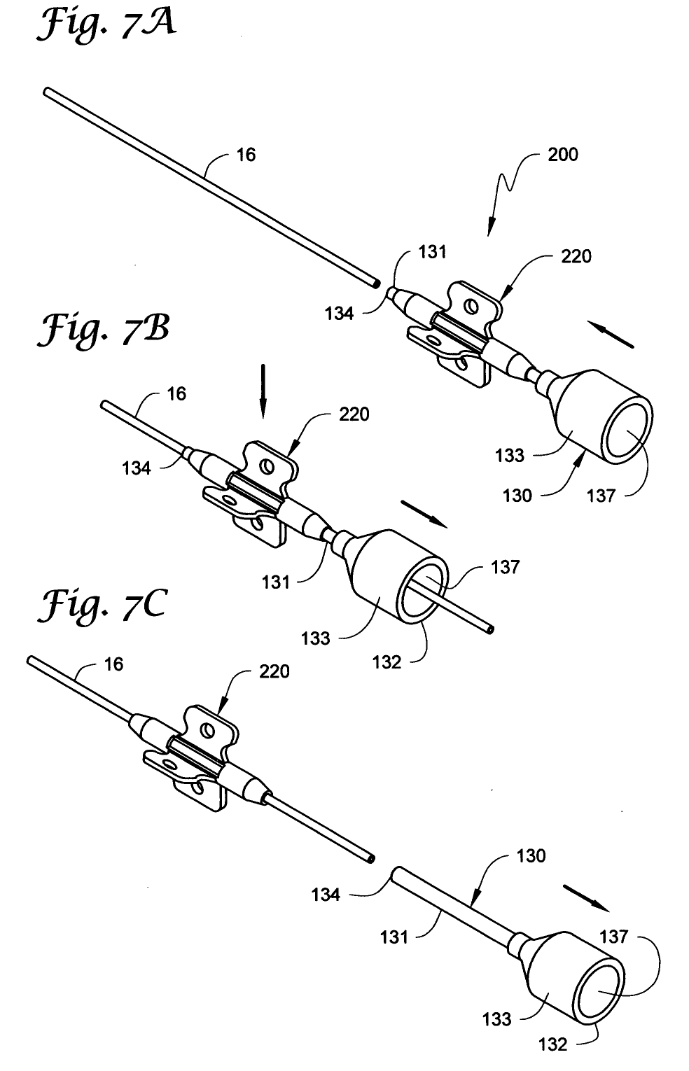

Yet further, FIGS. 7A-7C illustrate yet another embodiment of a method using an anchor system 200 to secure the catheter 16 relative to tissue of a patient. The anchor system 200 includes another embodiment of an anchor 220 that may be used to secure the catheter 16 to tissue.

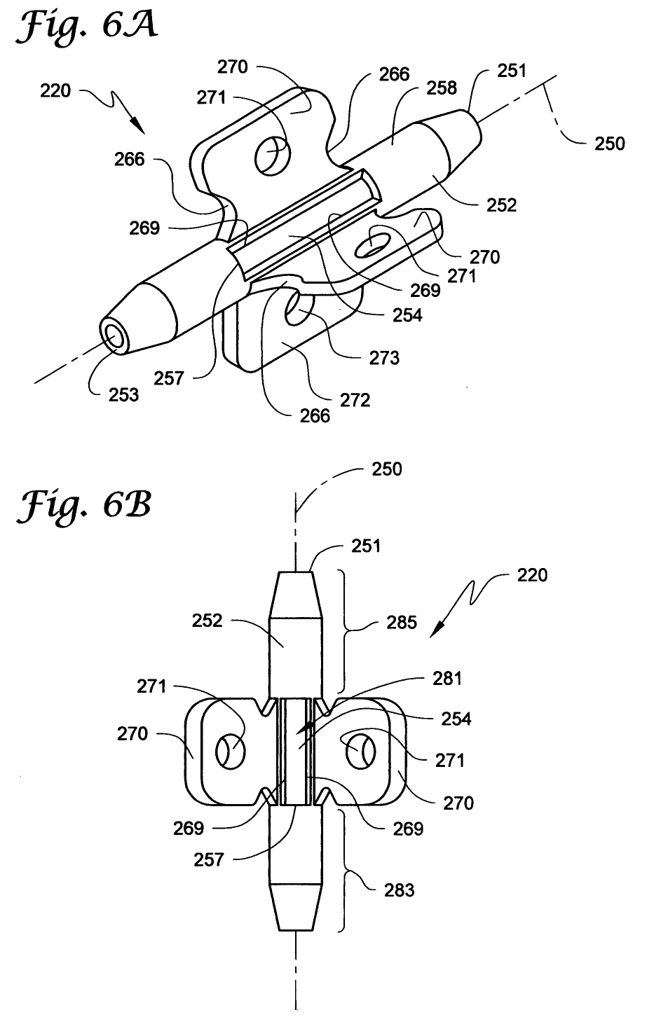

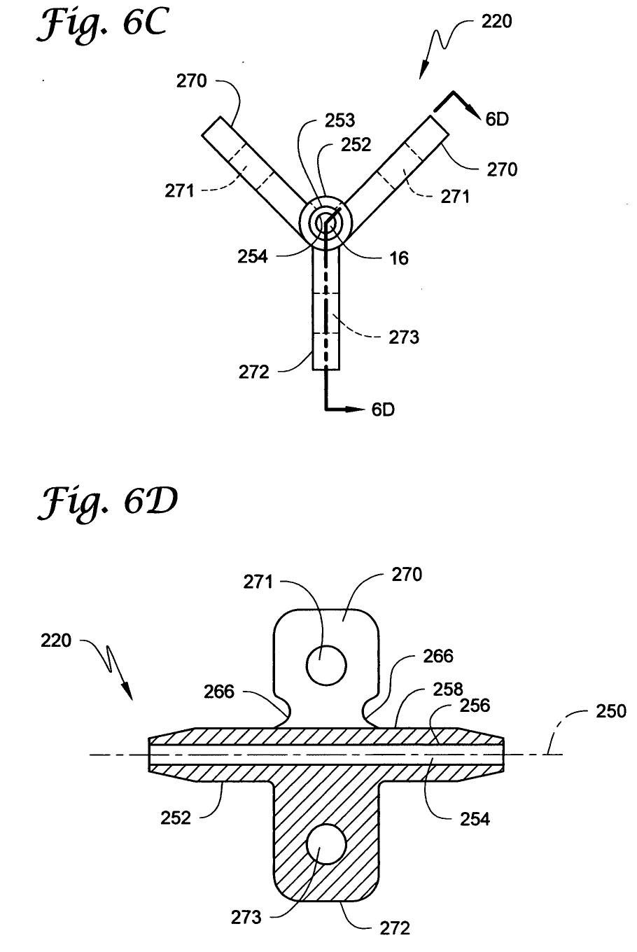

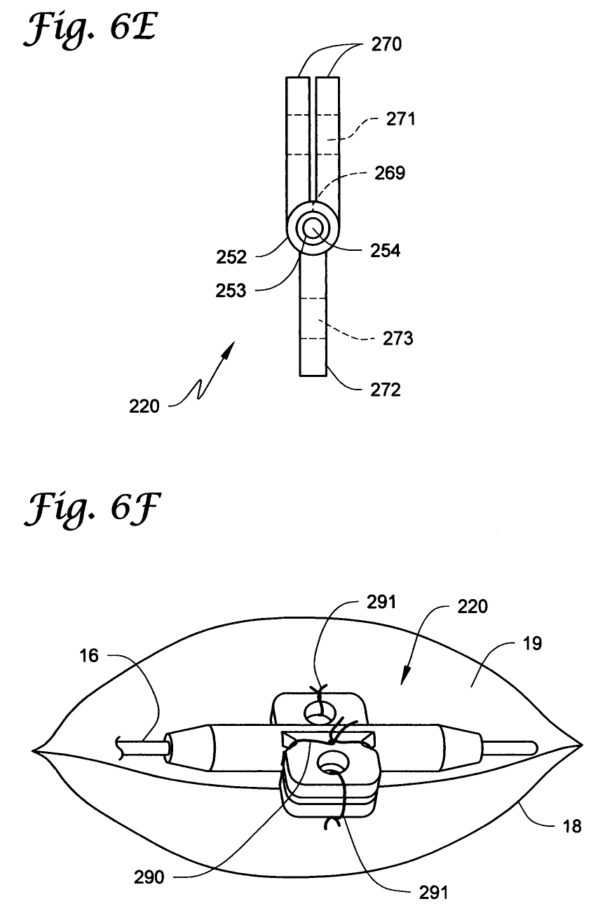

The anchor 220 is shown in further detail in FIGS. 6A-6F. FIG. 6A shows a perspective view of the anchor 220, FIG. 6B shows a plan view of the anchor 220, FIG. 6C shows an end view of the anchor 220, FIG. 6D shows a cross-section view taken along line D-D of FIG. 6C, FIG. 6E shows an end view of the anchor 220 with wings thereof brought together to secure a catheter 16 therein, and FIG. 6F shows a perspective view of the anchor 220 sutured in a desired site.

As shown in FIGS. 6A-6D, the anchor 220 includes an elongated body member 252 that extends between a first end 251 and a second end 253 along a centerline 250 of the anchor 220. When the anchor 220 is in a normal state (i.e., not flexed or expanded in any particular orientation or direction), the centerline 250 corresponds to a straight axis along which the elongated body member 252 extends.

The elongated body member 252 includes an inner surface 256 at a radial distance from the centerline 250 that defines a channel 254 from the first end 251 to the second end 253 along the centerline 250. The catheter 16 is positioned and secured to the anchor 220 within the channel 254 as will be further described herein. When the anchor 220 is in a normal state (i.e., not flexed or expanded in any particular direction or orientation), the channel 254 takes the form of a cylindrical channel. In at least one embodiment, the channel 254 has a cross-sectional inner diameter along the centerline 250 that is just slightly larger than the outer diameter of the catheter 16 intended to be secured by the anchor 220. For example, the inner diameter of the channel 254 may be 10 percent to 30 percent larger than the outer diameter of the catheter 16. However, in one or more other embodiments, the channel 254 has a cross-sectional inner diameter along the centerline 250 that is smaller than the outer diameter of the catheter 16 intended to be secured by the anchor 220. For example, the inner diameter of the channel 254 may be 20 percent to 50 percent smaller than the outer diameter of the catheter 16.

In at least one embodiment, the channel 254 has a cross-sectional inner diameter along the centerline 250 that is less than the outer diameter of a cylindrical surface of a catheter anchor placement tool (e.g., tool 130) as further described herein.

In other words, at least in one embodiment, the catheter 16 may be inserted through the channel 254 defined by the anchor 220 when the anchor 220 is in a normal state (e.g., when the inner diameter of channel 254 is slightly larger than the outer diameter of the catheter 16). However, such insertion is generally not easily accomplished due to the flexibility of the anchor and flexibility of the catheter.

The anchor 220 is formed of a flexible material such that, at least in the embodiment described with reference to FIG. 6, the flexible material is suitable to allow expansion of the diameter of channel 254 in the manner described herein (e.g., when loaded on an anchor placement tool), and which allows subsequent return to its normal state.

The elongate body member 252 of the anchor 220 includes an outer surface 258. A pair of wings 270 extends from a center region 281 of the outer surface 258 of the elongate body member 252. A slot 257 is formed in the outer surface 258 to the channel 254. At least in one embodiment, the slot 257 is elongated in the direction of the centerline 250 and includes side edges 269 that form an angle to each other. In one embodiment, bringing the wings 270 together causes the side edges 269 to come into contact (e.g., full surface contact) with each other. The pair of wings 270 are sufficiently separated in the normal state so that as the pair of wings 270 are brought together there is sufficient compression of the anchor 220 on the catheter 16 to adequately fictionally hold the catheter 16 within the anchor 220 (e.g., provide a compression fit).

The wings 270 extend substantially radially from the outer surface 258 along radials from the centerline 250. The wings 270 are generally planar with the planes being substantially planar to the longitudinal centerline 250. As shown in FIG. 6C, at least in one embodiment, wings 270 form an acute angle to each other (e.g., the wings form a V shape). Generally, the wider the wings (i.e., width being along the length of the centerline 250), combined with the length of slot 257, the greater the area of compression contact of the anchor 220 with the catheter 16. Further details with respect to at least portions of this anchor configuration is provided in U.S. Pat. No. 6,554,802 issued 29 Apr. 2003 to Pearson et al., entitled “Medical catheter anchor” which is incorporated by reference herein. It will be apparent that one or more of the features described therein may be used in combination with any of the anchor embodiments described herein.

Each of the wings 270 define a suture eyelet 271 (e.g., suture rings or openings) and a pair of opposed suture indents 266. The suture indents 266 extend into the side wall of the wings 270 for use in suturing the wings together. When the wings 270 are brought together, they cooperate with slot 257 to change the dimensions of the anchor (e.g., the inner diameter of the channel 254) to cause a compression force on a catheter 16 positioned therein. Further, when brought together, the suture eyelets 271 of each wing also come together to form a single suture eyelet 271 for securing the anchor 220 to tissue. Yet further, the pair of wings 270 provide a “handle” that allows a user to grasp and manipulate or hold the anchor 220.

Further, the anchor 220 includes an additional wing 272 (e.g., suture eyelet containing element) extending from the elongate body member 252 at the center region 281 of the elongate body member 252. The additional wing 272 is located in a plane parallel to the centerline 250 of the anchor 220. The wing 272 defines an additional suture eyelet 273 (e.g., suture ring or opening) that is offset from the centerline 250 of the anchor 220. As shown in FIG. 6, the additional suture eyelet 273 lies in a plane that is parallel to the centerline 250 extending through the anchor 220. In such an orientation, suturing of the anchor 220 to tissue is generally performed in an orthogonal manner relative to the centerline 250 of the channel 254. This is also the case for the eyelet 271 created when the wings 270 are brought together. As described with reference to the anchor 20 of FIG. 2, the suture eyelets (e.g., location points for suturing) may be provided at any location as long as such locations are offset from the centerline of the anchor.

The elongated body member 252 includes at least a portion 283 and/or 285 thereof along the length of the anchor 220 that is insertable into the tissue of the patient. The length of the portion 283 and/or 285 insertable into the tissue of the patient (e.g., extending from the pair of wings 270 to the first end 251 or second end 253 of the anchor 220) is, at least in one embodiment, greater than one (1) centimeter. Still further, such a length for insertion into the tissue may be greater than two (2) centimeters.

Still further, the elongated body member 252 of the anchor 220 includes an outer surface 258 that is tapered to provide regions thereof that are narrower at the first end 251 and second end 253 relative to the rest of the elongated body member 252. Such tapering may reduce the insertion force necessary to insert the anchor 220 into the tissue and further may act as a strain relief for the catheter 16 when secured therein.

With the pair of wings 270 and the additional wing 272 extending from the body member 252 at the center region 281 thereof, the two end portions 283, 285 of the elongated body member 252 can be configured in substantially the same manner. For example, as shown in FIG. 6, first end portion 285 between the pair of wings 270 and the first end 251 is configured the same as the second end portion 283 of the body member 252 between the pair of wings 270 and the second end 253. As the end portion 283 and end portion 285 are identical, a user cannot mistakenly load the anchor 220 on a catheter anchor placement tool incorrectly.

With further reference to the anchor 220 shown in FIG. 6, and FIGS. 7A-7C which show one illustrative embodiment of using this anchor 220 to secure catheter 16 relative to tissue of a patient, the anchor system 200 shall be further described. In addition to anchor 220, the anchor system 200 further includes a catheter anchor placement tool 130 that is substantially the same as the tool 130 described with reference to FIGS. 3-4. For example, the lumen 137 defined through the catheter placement tool 130 has a diameter that is greater than an outer diameter of the catheter 16 such that the catheter is allowed to pass through the tool 130. The cylindrical body portion 131 further includes an outer cylindrical surface 138 located between the first end 132 and the second end 134. The outer cylindrical surface 138 may be along substantially the entire length of the cylindrical body portion 131 or a certain defined length thereof. In this embodiment described with reference to FIGS. 6 and 7, the outer cylindrical surface 138 has an outer diameter that is greater than the cross-sectional inner diameter of the channel 254 defined by the elongated body member 252 of the anchor 220 (e.g., the inner diameter of the channel 254 may be just slightly larger or smaller than the outer diameter of the catheter 16).

In this illustrative embodiment of securing the catheter 16, the catheter 16 is positioned in the body as desired prior to positioning of the anchor 220 on the catheter 16, as represented generally by FIG. 7A. For example, at least in one embodiment, a needle device such as that described with reference to FIG. 3, may be inserted into the body at a desired location to position the catheter 16 or the tip thereof at the target site (e.g., spinal area). The catheter 16 may then be inserted into the lumen of the needle device and the tip of the catheter 16 positioned as desired at the target site. The needle device may then be withdrawn and removed from the body of the patient resulting in the catheter 16 being positioned, but unsecured.

Further, as shown in FIG. 7A, the cylindrical body portion 131 that includes the outer cylindrical surface 138 is inserted in the channel 252 defined in the anchor 220. With the outer diameter of the outer cylindrical surface 138 being greater than the cross-sectional inner diameter of the channel 254, the flexible anchor 220 is expanded into a flexed state (e.g., an expanded diameter state, different than the normal diameter state of the anchor).

With the anchor 220 loaded onto the cylindrical body portion 131 such that it is in a flexed state, an incision is made proximate the positioned catheter 16 to access an incision region (e.g., a tissue region, such as fascia or other connective tissue) of the body in a location where the anchor is to be secured. The positioned catheter 16 is then inserted into the cylindrical body portion 131 having the anchor 220 loaded thereon as shown in FIG. 7B.

The anchor 220 may then be advanced along the length of the catheter 16 until the desired position for the anchor 220 is reached (e.g., with at least one centimeter of portion 283 or 285 of the anchor 220 extending into the tissue, such as connective tissue (e.g., fascia)).

The anchor 220 may then be held in the desired place and the cylindrical body portion 131 withdrawn and removed from the body of the patient as shown in FIG. 7C. As the anchor 220 is removed from the cylindrical body portion 131, the anchor 220 will tend back to its normal state such that the inner diameter of the channel 254 will decrease.

A user then brings the pair of wings 270 together to provide a compression fit between the anchor 220 and the catheter 16 in the channel 254 as shown in FIG. 6E. As shown in FIG. 6F, the wings 270 are then sutured together using the indents 266 in the side walls thereof by placing a suture 290 around the indents 266 and pulling the knot so that the wings 270 are in contact with each other. With the wings 270 in contact, the slot edges 269 are also in contact and a compression fit of the anchor 220 on the catheter is provided.

Further as shown in FIG. 6F, the anchor 220 may then be sutured to connective tissue at the desired site. For example, each of the suture eyelets 271 and 273 of the anchor 220 offset from the centerline 250 thereof may sutured to the connective tissue using sutures 291 to secure the anchor 220 to the tissue.

Yet further, FIGS. 8A-8D illustrate yet another embodiment of an anchor 320 that may be used to secure the catheter 16 to tissue in much the same manner as described with reference to FIGS. 6 and 7. FIG. 8A shows a top plan view of the anchor 320, FIG. 8B shows an end view of the anchor 320, FIG. 8C shows a cross-section view taken along line C-C of FIG. 8A, and FIG. 8D shows an enlarged view of section D of FIG. 8C.

As shown in FIGS. 8A-8D, the anchor 320 includes an elongated body member 352 that extends between a first end 351 and a second end 353 along a centerline 350 of the anchor 320. The elongated body member 352 includes an inner surface 356 at a radial distance from the centerline 350 that defines a channel 354 from the first end 351 to the second end 353 along the centerline 350. Just as described with reference to the embodiment of FIGS. 6 and 7, when the anchor 320 is in a normal state, the channel 354 takes the form of a cylindrical channel.

In at least one embodiment, the channel 354 has a cross-sectional inner diameter along the centerline 350 that is just slightly larger than the outer diameter of the catheter 16 intended to be secured by the anchor 320. Further, the channel 354 has a cross-sectional inner diameter along the centerline 350 that is less than the outer diameter of a cylindrical surface of a catheter anchor placement tool (e.g., tool 130). In other words, at least in one embodiment, the catheter 16 can be inserted through the channel 354 defined by the anchor 320 when the anchor 320 is in a normal state. The anchor 320 is formed of a flexible material such that, at least in the embodiment described with reference to FIG. 8, the flexible material is suitable to allow expansion of the diameter of channel 354, such as when loaded on an anchor placement tool, and which allows subsequent return to its normal state.

The elongate body member 352 of the anchor 320 includes an outer surface 358. A pair of wings 370 extends from the outer surface 358 of the elongate body member 352. A slot 357 is formed in the outer surface 358 to the channel 354. Further, the anchor 320 includes an additional wing 372 (e.g., suture eyelet containing element) extending from the elongate body member 352.

The anchor 320 shown in FIGS. 8A-8D include features that are substantially the same as features of the anchor 220 described with reference to FIGS. 6 and 7. Such features including the pair of wings 370 and additional wing 372 operate in substantially the same manner as previously described such that bringing the wings 370 together provides a sufficient compression force to adequately frictionally hold the catheter 16 within the anchor 320 (e.g., provide a compression fit). As such, operation and implant of the anchor 320 shall not be repeated herein.

However, various differences exist between anchor 320 and anchor 220 described with reference to FIGS. 6 and 7. For example, the wings 370 extend substantially radially from a region of the elongate body member 352 that is closer to the first end 351 thereof. This results in an extended length between the wings 370 and the second end 353 of the elongate body member 352 that can be inserted into tissue when implanted. In at least one embodiment, this length may be greater than one (1) centimeter. Still further, such a length may be even greater than two (2) centimeters.

Further, various stability structures are provided on the outer surface 358 of the extended length portion of the elongated body member 352 between the wings 370 and the second end 353. One will recognize that various types of structures may be used to provide further stability of anchor 320 relative to the tissue when inserted therein. As shown in FIG. 8, for example, such stability structures take the form of anchor bumps.

Yet further, as shown in FIGS. 8C and 8D, the channel 354 includes a first portion 390 that has a slightly smaller diameter X than a second region 391 which has a diameter Y. The first portion 390 is adjacent the wings 370 of the anchor 320. Such a difference in diameter is beneficial in that insertion and removal of the catheter is easier and the holding of the catheter is concentrated in the region of the wings.

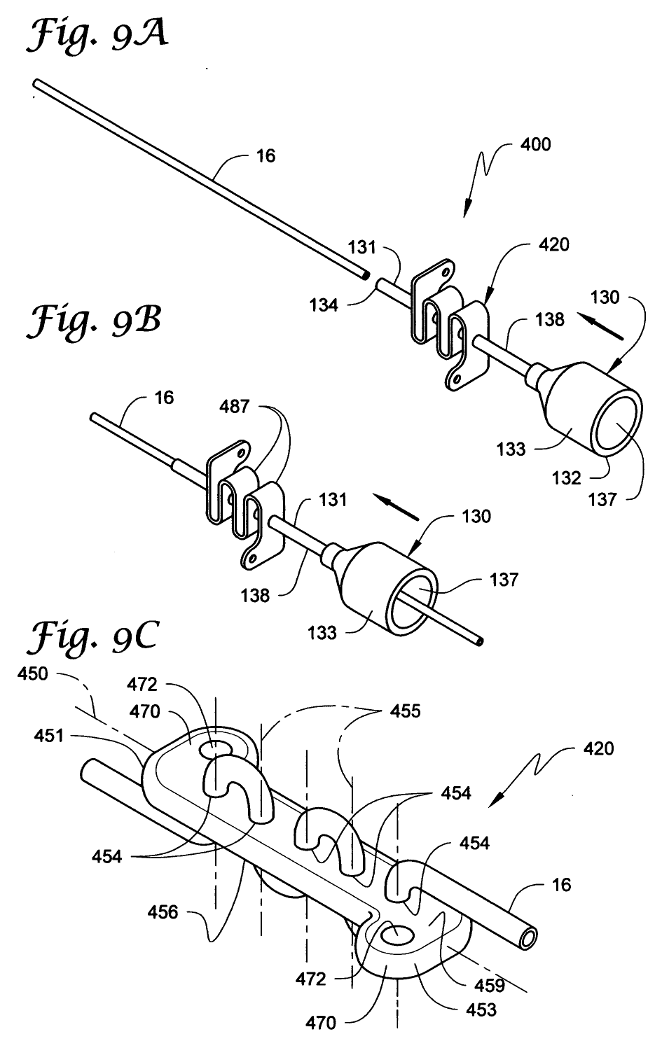

Yet further, FIGS. 9A-9C illustrate yet another embodiment of a method using an anchor system 400 to secure the catheter 16 relative to tissue of a patient. The anchor system 400 includes one embodiment of an anchor 420 that may be used to secure the catheter 16 to tissue. The anchor 420 is shown in FIG. 9C in a perspective view, wherein the anchor is in a normal state having a catheter 16 secured therein. Further, FIGS. 9B-9C show perspective views of the anchor 420 in a flexed stated loaded on a catheter anchor placement tool 130.

The anchor 420, as shown in its normal state in FIG. 9C, includes a generally planar elongated body member 452 that extends between a first end 451 and a second end 453 along a centerline 450 of the anchor 420. When the anchor 420 is in a normal state (i.e., not flexed or expanded in any particular orientation or direction), the centerline 450 corresponds to a straight axis along which the elongated body member 452 extends.

The elongated planar body member 452 includes a lower surface 456 and an upper surface 458. A plurality of openings 454 are spaced along the centerline 450 of the elongated planar body member 452. Each of the openings 454 extends from the lower surface 456 to the upper surface 458 along an axis 455 that is orthogonal to the centerline 450 in the normal state.

When the anchor 420 is in a normal state and without the catheter 16 secured therein, each of the openings 454 may have a diameter that is less than the outer diameter of the catheter 16 or slightly more than the diameter of the catheter 16. In other words, at least in one embodiment, the catheter 16 cannot be inserted through the openings 420 but in other embodiments, it may be inserted when the anchor 420 is in a normal state. The anchor 420 is formed of a flexible material such that, at least in the embodiment described with reference to FIG. 9, the flexible material is suitable to allow for the elongated planar body member 452 to be folded upon itself when loaded on an anchor placement tool 130 in a flexed state such that the openings 454 align along an axis parallel to the centerline 450.

Further, the anchor 420 includes suture eyelet containing elements 470 extending from the elongated planar body member 452 at each of the ends 451 and 453 thereof. The suture eyelet containing elements 470 are located in a plane parallel to the centerline 450 of the anchor 420. The suture eyelet containing elements 470 define two suture eyelets 472 (e.g., suture rings or openings) that are each offset from the centerline 450 of the anchor 420. As shown in FIG. 9, each of the suture eyelets 472 lie in a plane that is parallel to the centerline 450 extending through the anchor 420. As described with reference to the anchor 20 of FIG. 2, the suture eyelets 472 (e.g., location points for suturing) may be provided at any location as long as such locations are offset from the centerline of the anchor.

With further reference to FIGS. 9A-9C which shows one illustrative embodiment of using this anchor 420 to secure catheter 16 relative to tissue of a patient, the anchor system 400 shall be further described. In addition to the anchor 420, the anchor system 400 further includes a catheter anchor placement tool 130 that is substantially the same as the tool 130 described with reference to FIGS. 3-4. For example, the lumen 137 defined through the catheter placement tool 130 has a diameter that is greater than an outer diameter of the catheter 16 such that the catheter is allowed to pass through the tool 130. The cylindrical body portion 131 further includes an outer cylindrical surface 138 located between the first end 132 and the second end 134. The outer cylindrical surface 138 may be along substantially the entire length of the cylindrical body portion 131 or a certain defined length thereof.

In this embodiment described with reference to FIG. 9, the outer cylindrical surface 138 has an outer diameter that is greater than the diameter of one or more of the openings 454 defined by the elongated planar body member 452 of the anchor 420. In one or more embodiments, the diameters may be about the same size, or even the diameter of the openings 454 may be slightly larger than the outer diameter of the outer cylindrical surface 138.

In this illustrative embodiment of securing the catheter 16, the catheter 16 is positioned in the body as desired prior to positioning of the anchor 420 on the catheter 16, as represented generally by FIG. 9A. For example, at least in one embodiment, a needle device such as that described with reference to FIG. 3, may be inserted into the body at a desired location to position the catheter 16 or the tip thereof at the target site (e.g., spinal area). The catheter 16 may then be inserted into the lumen of the needle device and the tip of the catheter 16 positioned as desired at the target site. The needle device may then be withdrawn and removed from the body of the patient resulting in the catheter 16 being positioned, but unsecured.

Further, as shown in FIG. 9A, the cylindrical body portion 131 that includes the outer cylindrical surface 138 is inserted through the openings 454 defined in the anchor 420 as the elongated planar body member 452 is flexed and folded upon itself (e.g., resulting in a number of folds 487). One will recognize that the number of openings 454 and the number of folds may vary, and that the present invention is not limited to the use of an particular number shown or described herein. With the elongated planar body member 452 flexed and folded upon itself, the flexible anchor 420 in its flexed state is held on the cylindrical body portion 131 by various forces, including those exerted by the folds tending to return to the normal state.

With the anchor 420 loaded onto the cylindrical body portion 131 such that it is in a flexed state, an incision is made proximate the positioned catheter 16 to access an incision region (e.g., a tissue region, such as fascia or other connective tissue) of the body in a location where the anchor 420 is to be secured. The positioned catheter 16 is then inserted into the cylindrical body portion 131 having the anchor 420 loaded thereon as shown in FIG. 9B.

The anchor 420 may then be advanced along the length of the catheter 16 until the desired position for the anchor 420 is reached. The anchor 420 may then be held in the desired place and the cylindrical body portion 131 withdrawn and removed from the body of the patient resulting in the elongated planar body member 452 returning to the normal state with the catheter 16 secured therein as shown in FIG. 9C. With the threading of the catheter 16 through the multiple openings 454, the catheter is secured in the anchor 420.

The anchor 420 may then be sutured to connective tissue at the desired site. For example, each of the suture eyelets 470 of the anchor 420 offset from the centerline 450 thereof may be sutured to the connective tissue to secure the anchor 420 to the tissue.

The complete disclosure of the patents, patent documents, and publications cited in the Background, the Detailed Description of Exemplary Embodiments, and elsewhere herein are incorporated by reference in their entirety as if each were individually incorporated.

Illustrative embodiments of this invention are discussed and reference has been made to possible variations within the scope of this invention. These and other variations, combinations, and modifications in the invention will be apparent to those skilled in the art without departing from the scope of the invention, and it should be understood that this invention is not limited to the illustrative embodiments set forth herein. Accordingly, the invention is to be limited only by the claims provided below and equivalents thereof.

What is claimed is:

1. An anchor system for use in securing a catheter to tissue, the system comprising:
a catheter anchor placement tool comprising an elongate body portion, wherein the elongate body portion comprises a non-flexible outer cylindrical surface located between a first end and a second end of the elongate body portion, and further wherein the elongate body portion defines a lumen through the elongate body portion from the first end to the second end having a diameter that is greater than an outer diameter of the catheter to allow the catheter to pass through the catheter anchor placement tool; and
an anchor comprising a body member formed of a flexible material defining at least one opening through which the catheter is positioned when secured using the anchor, wherein the outer cylindrical surface of the catheter anchor placement tool is configured so as to hold the anchor thereon such that the inner diameter of the at least one opening defined through the anchor is greater than the outer diameter of the catheter when the anchor is positioned on the outer cylindrical surface.

2. The anchor system of claim 1, wherein the body member of the anchor comprises an elongate body member that defines a channel extending through the elongate body member from a first end to a second end thereof, wherein the cross-sectional inner diameter along at least a portion of the length of the channel is less than the outer diameter of the catheter when in a normal state, wherein the outer cylindrical surface of the catheter anchor placement tool has an outer diameter sized to hold the anchor thereon in a flexed state such that the cross-sectional inner diameter of at least a portion of the channel is greater than the outer diameter of the catheter when the anchor is positioned on the outer cylindrical surface.

3. The anchor system of claim 2, wherein the cross-sectional inner diameter along the entire length of the channel is less than the outer diameter of the catheter when in a normal state.

4. The anchor system of claim 1, wherein the anchor further comprises at least one suture eyelet containing element extending from the body member, wherein the at least one suture eyelet containing element defines at least two suture eyelets, each of the at least two suture eyelets being offset from a centerline extending through the anchor to provide at least two separate suture points offset from the centerline for suturing the anchor to tissue.

5. The anchor system of claim 4, wherein each of the at least two suture eyelets lie in a plane that is orthogonal to the centerline extending through the anchor.

6. The anchor system of claim 4, wherein each of the at least two suture eyelets lie in a plane that is parallel to the centerline extending through the anchor.

7. The anchor system of claim 4, wherein the anchor comprises at least one suture eyelet containing element extending from the body member at a position along the length of the body member, wherein the distance along the length of the body member from the position of the at least one suture eyelet containing element to at least one of the first and second ends of the body member is at least 1 cm and is configured for insertion into tissue.

8. The anchor system of claim 4, wherein the body member of the anchor further comprises one or more stability structures positioned between the at least one suture eyelet containing element and at least one of the first end and second end of the body member.

9. The anchor system of claim 4, wherein the anchor comprises at least one suture eyelet containing element extending from the body member at a center region of the body member, wherein the portion of the body member between the position of the at least one suture eyelet containing element and the first end is configured the same as the portion of the body member between the suture eyelet containing element and the second end.

10. The anchor system of claim 2, wherein the elongate body member comprises a slot that extends from an outer surface of the elongate body member to the channel defined thereby, the slot having side edges opposed to each other, wherein the anchor further comprises a pair of wings extending from the elongate body member on opposite sides of the slot, the pair of wings separated from each other by an angle, wherein the wings and the slot cooperate so that when the wings are brought into contact with each other the anchor secures the catheter in the channel, wherein the pair of wings define a first suture eyelet offset from a centerline of the channel when the pair of wings are brought into contact with each other, and further wherein the anchor comprises an additional wing extending from the elongate body member defining an additional suture eyelet offset from the centerline of the channel.

11. The anchor system of claim 1, wherein the body member of the anchor comprises a flexible planar body member lying along a plane when in a normal state, wherein the planar body member defines a plurality of openings therein to receive the outer cylindrical surface of the catheter anchor placement tool when the planar body member is folded upon itself in a flexed state.

12. The anchor system of claim 1, wherein the catheter anchor placement tool comprises a needle device, wherein the needle device comprises a needle shaft comprising the outer cylindrical surface and defining at least a portion of the lumen, wherein the needle shaft terminates in a needle tip for insertion into the body of a patient.

13. The anchor system of claim 1, wherein the catheter anchor placement tool comprises:
a cylindrical portion comprising the outer cylindrical surface and defining at least a portion of the lumen; and
a holding portion coupled to the cylindrical portion and defining a further portion of the lumen continuous with the portion of the lumen defined by the cylindrical portion.

14. An anchor for use in securing a catheter to tissue, the anchor comprising an elongate body member, wherein a channel is defined through the elongate body member from a first end to a second end thereof, wherein the cross-sectional inner diameter along at least a portion of the length of the channel is less than the outer diameter of the catheter when the anchor is in a normal state preventing the insertion of the catheter through the channel, and further wherein the anchor comprises a flexible material that allows the cross-sectional inner diameter along the portion of the length of the channel to be expanded to a cross-sectional inner diameter greater than the outer diameter of the catheter when the anchor is in a flexed state.

15. The anchor of claim 14, wherein the cross-sectional inner diameter along the entire length of the channel is less than the outer diameter of the catheter when in a normal state.

16. The anchor of claim 14, wherein the anchor further comprises at least one suture eyelet containing element extending from the elongate body member, wherein the at least one suture eyelet containing element defines at least two suture eyelets, each of the at least two suture eyelets being offset from a centerline extending through the channel to provide at least two separate suture points offset from the centerline for suturing the anchor to tissue.

17. The anchor of claim 16, wherein each of the at least two suture eyelets lie in a plane that is orthogonal to the centerline extending through the channel.

18. The anchor of claim 16, wherein each of the at least two suture eyelets lie in a plane that is parallel to the centerline extending through the channel.

19. The anchor of claim 16, wherein the anchor comprises a suture eyelet containing element extending from the elongate body member at a position along the length of the elongate body member, wherein the distance along the length of the elongate body member from the position of the at least one suture eyelet containing element to at least one of the first and second ends of the body member is at least 1 cm and is configured for insertion into tissue.

20. The anchor of claim 16, wherein the anchor comprises one or more stability structures positioned between the at least one suture eyelet containing element and at least one of the first and the second ends of the elongate body member.

21. The anchor of claim 16, wherein the anchor comprises at least one suture eyelet containing element extending from the elongate body member at a center region along the length of the elongate body member, wherein the portion of the elongate body member between the position of the at least one suture eyelet containing element and the first end is configured the same as the portion of the elongate body member between the suture eyelet containing element and the second end.

22. An anchor for use in securing a catheter to tissue, the anchor comprising:
an elongate body member, wherein a channel is defined through the elongate body member from a first end to a second end thereof, wherein the elongate body member comprises a slot that extends from an outer surface of the elongate body member to the channel defined thereby, the slot having side edges opposed to each other;
a pair of wings extending from the elongate body member on opposite sides of the slot, the pair of wings separated from each other by an angle, wherein the wings and the slot cooperate so that when the wings are brought into contact with each other the anchor secures the catheter in the channel, wherein the pair of wings define a first suture eyelet offset from a centerline of the channel when the pair of wings are brought into contact with each other; and
an additional wing extending from the elongate body member defining an additional suture eyelet offset from the centerline of the channel.

23. The anchor of claim 22, wherein the pair of wings extend from the elongate body member at a position along the length of the elongate body member, wherein the distance along the length of the elongate body member from the position of the pair of wings to at least one of the first and second ends of the body member is at least 1 cm and is configured for insertion into tissue.

24. The anchor of claim 23, wherein the distance is at least 2 cm.

25. The anchor of claim 22, wherein the pair of wings extend from the elongate body member proximate the first end of the elongate body member, and further wherein the elongate body member comprises one or more stability structures between the pair wings and the second end of the elongate body member.

26. The anchor of claim 22, wherein the pair of wings extend from the elongate body member at a center region between the first end and the second end of the elongate body member, and further wherein the portion of the elongate body member between the pair of wings and the first end is configured the same as the portion of the elongate body member between the pair of wings and the second end.

27. A method of securing a catheter to tissue, wherein the method comprises:
providing an anchor comprising a body member, wherein the body member comprises a flexible material defining at least one opening through the body member;
inserting a non-flexible portion of a catheter anchor placement tool into the at least one opening of the anchor, wherein the anchor is held thereon;
inserting the catheter through a lumen in the catheter placement tool; and
holding the anchor and removing the catheter anchor placement tool so as to position the anchor on the catheter.

28. The method of claim 27, wherein providing the anchor comprises providing an elongate body member defining a channel through the elongate body member from a first end to a second end thereof, wherein the cross-sectional inner diameter along a least a portion of the length of the channel is less than the outer diameter of the catheter in a normal state when apart from the catheter anchor placement tool, wherein inserting the non-flexible portion of the catheter anchor placement tool into the channel of the anchor comprises expanding the cross-sectional inner diameter along at least a portion of the length of the channel to a cross-sectional inner diameter that is greater than the outer diameter of the catheter.

29. The method of claim 28, wherein holding the anchor and removing the catheter anchor placement tool so as to position the anchor on the catheter comprises holding the anchor and removing the catheter anchor placement tool so as to provide a compression fit of the anchor on the catheter as the cross-sectional inner diameter of the channel returns to the normal state.

30. The method of claim 27, wherein holding the anchor and removing the catheter anchor placement tool so as to position the anchor on the catheter further comprises manipulating one or more portions of the anchor to provide a compression fit of the anchor on the catheter.

31. The method of claim 27, wherein providing the anchor further comprises providing at least one suture eyelet containing element extending from the body member, wherein the at least one suture eyelet containing element defines at least two suture eyelets, each of the at least two suture eyelets offset from a centerline extending through the channel, and further wherein the method comprises placing a suture through each of the at least two suture eyelets and tissue to secure the anchor to the tissue.

32. The method of claim 27, wherein the method further comprises:
making an incision in a tissue region; and
inserting a portion of the anchor at least 1 cm into the tissue.

33. The method of claim 32, wherein inserting the portion of the anchor comprises inserting the portion of the anchor at least 2 cm into the tissue.

34. The method of claim 27, wherein inserting a catheter anchor placement tool into the at least one opening comprises loading the anchor onto a needle device, wherein the needle device comprises a needle shaft comprising an outer cylindrical surface and defining at least a portion of the lumen thereof, wherein the needle shaft terminates in a needle tip for insertion into the body of a patient, and further wherein, prior to inserting the catheter through the lumen, the method comprises:
making an incision in a region of the patient; and
inserting the needle device with the anchor loaded thereon through the incision.

35. The method of claim 27, wherein inserting a catheter anchor placement tool into the channel of the anchor comprises loading the anchor onto a cylindrical portion of the catheter anchor placement tool defining at least a portion of the lumen, and further wherein the method comprises:
providing a needle device comprising a needle shaft defining a lumen through the needle and terminating at a needle tip for insertion into the body of a patient;
inserting the needle device into the patient, wherein the catheter is inserted through the lumen in the needle device and positioned in the patient;
removing the needle device from the patient; and
making an incision in the patient proximate the positioned catheter such that the catheter can be inserted into the lumen of the catheter placement tool and the anchor positioned on the catheter at a desired location.
